(19) United States
(12) Patent Application Publication
(10) Pub. No.: US 2026/0351370 A1
(43) Pub. Date: Nov. 5, 2026

(54) SYSTEM AND METHOD FOR EXTRACTING STRUCTURED CLINICAL INFORMATION FROM A FREE-TEXT MEDICAL REPORT

(71) Applicants: Shaare Zedek Scientific LTD, Jerusalem (IL); Technion Research & Development Foundation Limited, Haifa (IL)

(72) Inventors: Dan TURNER, Jerusalem (IL); Gili FOCHT, Jerusalem (IL); Mordechay Pinchas FREIMAN, Haifa (IL); Liam HAZAN, Haifa (IL); Ruth CYTTER-KUINT, Jerusalem (IL); Talar Silvi HAGOPIAN, Jerusalem (IL)

(21) Appl. No.: 19/660,259

(22) Filed: Apr. 28, 2026

Related U.S. Application Data
(60) Provisional application No. 63/796,438, filed on Apr. 29, 2025.

(51) Int. Cl. G16H 15/00

(52) U.S. Cl. CPC G16H 15/00

(57) ABSTRACT

The presently disclosed subject matter discloses a system and method for extracting structured clinical information from a free-text medical report. The system and method include: (A) obtaining: (a) a text segment from the medical report written in at least one natural language, and (b) a machine learning model capable of (i) receiving a given text segment from a given medical report written in at least one given natural language, and (ii) providing given structured clinical information indicating presence or absence of given clinical phenotypes; and, (B) extracting, based on the text segment and utilizing the machine learning model, structured clinical information indicating presence or absence of clinical phenotypes.

### TECHNICAL FIELD

The present invention relates to the field of free-text medical reports, and more particularly, to systems and methods for extracting structured clinical information from such reports.

### BACKGROUND

Medical imaging, particularly through modalities such as Computed Tomography (CT) and Magnetic Resonance Imaging (MRI), plays a pivotal role in the management of complex conditions like Crohn's Disease (CD), Multiple Sclerosis (MS), etc. These imaging techniques form the foundation for diagnosis, disease monitoring, and guiding therapeutic decisions. The valuable clinical insights derived from such imaging are typically captured in medical reports, e.g., radiology reports, written in free-text format. While this form allows radiologists to convey nuanced observations, it poses significant challenges for systematic analysis, large-scale research, and integration with Electronic Health Records (EHRs).

Free-text medical reports are a rich but underutilized resource for computational methods due to their unstructured nature. Extracting structured phenotypes and clinically relevant information from these reports, especially in the context of diseases with high heterogeneity like CD, is both time-intensive and dependent on domain-specific expertise. The manual curation of such data is impractical for large-scale studies, especially when the goal is to assess inflammatory burden or model disease progression across populations.

To address these limitations, recent efforts have explored the application of Natural Language Processing (NLP) techniques, particularly those based on generative Large Language Models (LLMs) such as GPT-4. These models are capable of interpreting and converting complex medical narratives into structured data using free-text prompts, thus reducing reliance on annotated datasets. However, while these tools demonstrate strong performance in widely spoken languages like English, where training data is abundant, their effectiveness significantly drops in low-resource languages due to insufficient linguistic representation in training corpora.

Moreover, privacy concerns loom large when training models on sensitive medical text, as inadvertent exposure of protected health information (PHI) can compromise patient confidentiality. Another persistent issue is data imbalance, which is especially pronounced in medical datasets where rare but clinically critical conditions are underrepresented. This imbalance undermines the performance of fine-tuned models, leading to suboptimal detection of less common findings.

Consequently, while generative LLMs offer a promising avenue for automating the extraction of clinical insights from medical reports, particularly for diseases like CD, they remain constrained by limited language support, data privacy risks, the scarcity of annotated data, and difficulties in capturing rare clinical conditions. These challenges highlight the need for more robust, scalable, and inclusive NLP solutions capable of advancing large-scale disease research and improving clinical decision-making in diverse linguistic and healthcare settings.

Considering the above, there is a need in the art for a new system and method for extracting structured clinical information from a free-text medical report.

### GENERAL DESCRIPTION

In accordance with a first aspect of the presently disclosed subject matter, there is provided a method for extracting structured clinical information from a free-text medical report comprising: obtaining: (a) a text segment from the medical report written in at least one natural language, and (b) a machine learning model capable of (i) receiving a given text segment from a given medical report written in at least one given natural language, and (ii) providing given structured clinical information indicating presence or absence of given clinical phenotypes; and, extracting, based on the text segment and utilizing the machine learning model, structured clinical information indicating presence or absence of clinical phenotypes; wherein the machine learning model is pre-trained using labeled training data including pairs of text segments from historical medical reports, labeled as either “Match”, indicating that the pair is from the same medical report, or “Not Match”, indicating that the pair is not from the same medical report, and wherein, following the pre-training, the machine learning model is further trained to identify semantic agreement between text segments and prompts by evaluating which of two opposing queries, one indicating presence and one indicating absence of a specific clinical phenotype, yields a higher match probability.

In some cases, said machine learning model is pre-trained using a Section Matching Prediction (SMP) task.

In some cases, the method further comprises obtaining one or more prompts corresponding to one or more clinical queries related to one or more clinical phenotypes, wherein the machine learning model is configured to receive the one or more prompts, along with the text segment, and to provide the given structured clinical information based on the semantic agreement between the text segment and the one or more prompts, such that the structured clinical information is further based on the prompts.

In some cases, the one or more prompts are provided in a hierarchical fashion.

In some cases, prior to the SMP task, the machine learning model is pre-trained using a Masked Language Modeling (MLM) task, where one or more tokens in one or more training medical reports are replaced with one or more mask tokens, and the model is trained to predict the masked tokens based on surrounding contextual tokens.

In some cases, the medical report is a radiology report.

In some cases, the text segment is a “Findings” section.

In some cases, each pair of the pairs of text segments involves a first text segment, corresponding to a “Findings” section, and a second text segment, corresponding to an “Impression” section.

In some cases, the semantic is determined between a “Findings” section and a corresponding prompt.

In some cases, the at least one natural language is a low-resource language.

In some cases, the at least one natural language includes two or more languages.

In accordance with a second aspect of the presently disclosed subject matter, there is provided a system for extracting structured clinical information from a free-text medical report, the system comprising a processing circuitry configured to: obtain: (a) a text segment from the medical report written in a natural language, and (b) a machine learning model capable of (i) receiving a given text segment from a given medical report written in a given natural language, and (ii) providing given structured clinical information indicating presence or absence of given clinical phenotypes; and, extract, based on the text segment and the machine learning model, structured clinical information indicating presence or absence of clinical phenotypes; wherein the machine learning model is pre-trained using labeled training data including pairs of text segments from historical medical reports, labeled as either “Match”, indicating that the pair is from the same medical report, or “Not Match”, indicating that the pair is not from the same medical report, and wherein, following the pre-training, the machine learning model is further trained to identify semantic agreement between text segments and prompts by evaluating which of two opposing queries, one indicating presence and one indicating absence of a specific clinical phenotype, yields a higher match probability.

In some cases, said machine learning model is pre-trained using a Section Matching Prediction (SMP) task.

In some cases, the system further comprises obtaining one or more prompts corresponding to one or more clinical queries related to one or more clinical phenotypes, wherein the machine learning model is configured to receive the one or more prompts, along with the text segment, and to output the given structured clinical information based on the semantic agreement between the text segment and the one or more prompts, such that the structured clinical information is further based on the prompts.

In some cases, the one or more prompts are provided in a hierarchical fashion.

In some cases, prior to the SMP task, the machine learning model is pre-trained using a MLM task, where one or more tokens in one or more training medical reports are replaced with one or more mask tokens, and the model is trained to predict the masked tokens based on surrounding contextual tokens.

In some cases, the medical report is a radiology report.

In some cases, the text segment is a “Findings” section.

In some cases, each pair of the pairs of text segments involves a first text segment, corresponding to a “Findings” section, and a second text segment, corresponding to an “Impression” section.

In some cases, the semantic is determined between a “Findings” section and a corresponding prompt.

In some cases, the natural language is a low-resource language.

In some cases, the at least one natural language includes two or more languages.

In accordance with a second aspect of the presently disclosed subject matter, there is provided a non-transitory computer readable storage medium having computer readable program code embodied therewith, the computer readable program code, executable by at least one processor to perform a method for extracting structured clinical information from free-text medical reports, the method comprising: obtaining: (a) a text segment from the medical report written in a natural language, and (b) a machine learning model capable of (i) receiving a given text segment from a given medical report written in a given natural language, and (ii) providing given structured clinical information indicating presence or absence of given clinical phenotypes; and, extracting, based on the text segment and the machine learning model, structured clinical information indicating presence or absence of clinical phenotypes; wherein the machine learning model is pre-trained using labeled training data including pairs of text segments from historical medical reports, labeled as either “Match”, indicating that the pair is from the same medical report, or “Not Match”, indicating that the pair is not from the same medical report, and wherein, following the pre-training, the machine learning model is further trained to identify semantic agreement between text segments and prompts by evaluating which of two opposing queries, one indicating presence and one indicating absence of a specific clinical phenotype, yields a higher match probability.

### BRIEF DESCRIPTION OF THE DRAWINGS

In order to understand the presently disclosed subject matter and to see how it may be carried out in practice, the subject matter will now be described, by way of non-limiting examples only, with reference to the accompanying drawings, in which:

FIG. 1 illustrates an exemplary bar graph illustrating comparison of the median AUC and F1-score of three models (Standard Fine-tuning, SMP-BERT Zero-Shot, and SMP-BERT+tuning) over all phenotypes with 10+ positives. Error bars represent the Interquartile Range (IQR);

FIG. 2 illustrates exemplary input and output processed by the disclosed system and method, in accordance with the presently disclosed subject matter;

FIG. 3 illustrates an exemplary SMP-BERT methodology including three pre-training tasks, in accordance with the presently disclosed subject matter;

FIG. 4 illustrates an exemplary SMP-tuning process, which involves fine-tuning SMP-BERT by generating both a positive and a negative instance for each annotated example, in accordance with the presently disclosed subject matter;

FIG. 5 illustrates an exemplary flowchart of the study design, outlining the sequence of processing steps from data acquisition to model evaluation, in accordance with the presently disclosed subject matter;

FIG. 6 illustrates median F1 scores and interquartile ranges (IQRs) for SMP-BERT with SMP-tuning and standard fine-tuning, evaluated across varying training set sizes, in accordance with the presently disclosed subject matter;

FIG. 7 illustrates an exemplary an exemplary line chart plotting F1 scores against the number of positive instances for all phenotypes in the dataset (totaling 300), in accordance with the presently disclosed subject matter;

FIG. 8 illustrates an exemplary performance comparison table reporting F1 and AUC scores for each model across various phenotypes, in accordance with the presently disclosed subject matter;

FIG. 9 illustrates an exemplary table presenting the results of an ablation study on pre-training tasks, in accordance with the presently disclosed subject matter;

FIG. 10 illustrates a block diagram schematically illustrating one example of a system for extracting structured clinical information from a free-text medical report, in accordance with the presently disclosed subject matter; and,

FIG. 11 illustrates an exemplary flowchart illustrating an example of a sequence of operations carried out by a system for extracting structured clinical information from a free-text medical report, in accordance with the presently disclosed subject matter.

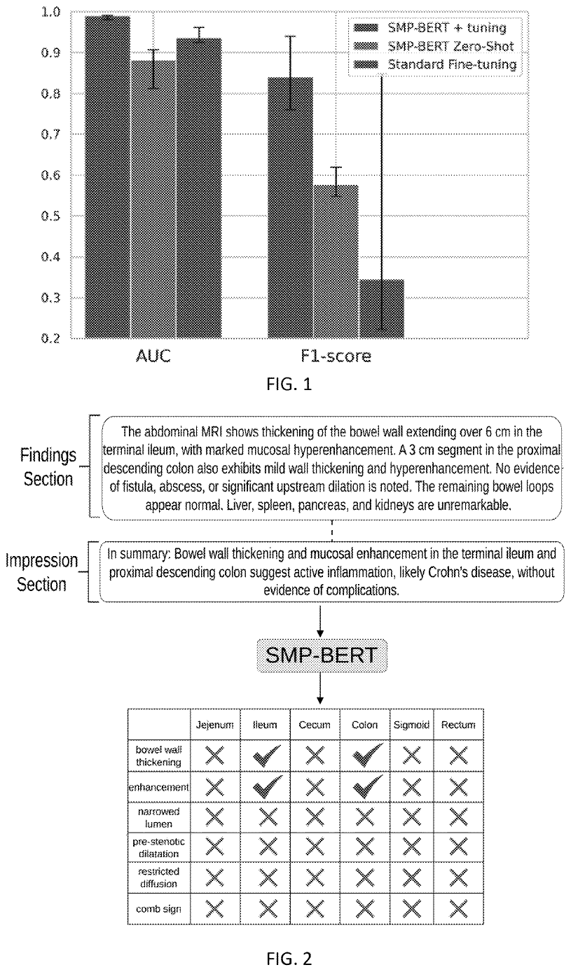

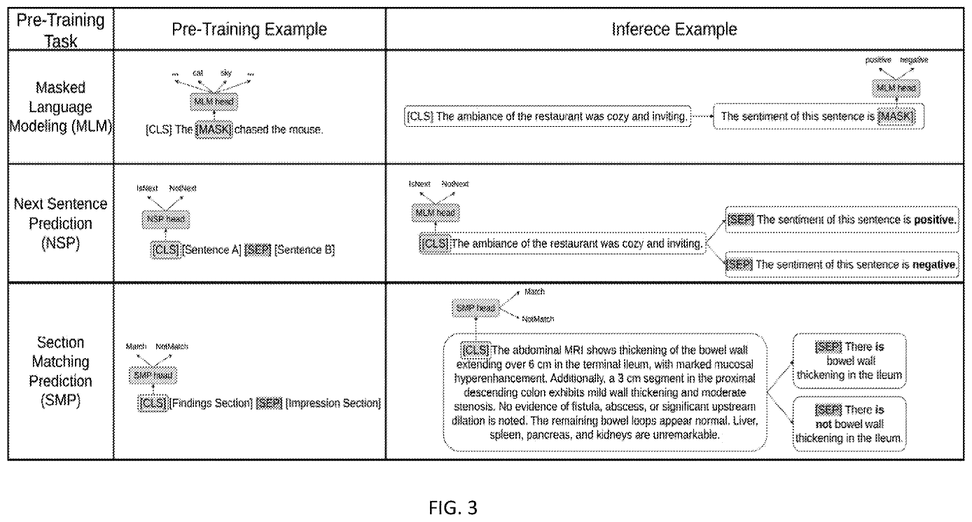

### DETAILED DESCRIPTION

In the following detailed description, numerous specific details are set forth in order to provide a thorough understanding of the presently disclosed subject matter. However, it will be understood by those skilled in the art that the presently disclosed subject matter may be practiced without these specific details. In other instances, well-known methods, procedures, and components have not been described in detail so as not to obscure the presently disclosed subject matter.

In the drawings and descriptions set forth, identical reference numerals indicate those components that are common to different embodiments or configurations.

Unless specifically stated otherwise, as apparent from the following discussions, it is appreciated that throughout the specification discussions utilizing terms such as “obtaining”, “extracting”, “providing”, “receiving”, “training”, or the like, include action and/or processes of a computer that manipulate and/or transform data into other data, said data represented as physical quantities, e.g., such as electronic quantities, and/or said data representing the physical objects. The terms “computer”, “processor”, “processing resource”, “processing circuitry”, and “controller” should be expansively construed to cover any kind of electronic device with data processing capabilities, including, by way of non-limiting example, a personal desktop/laptop computer, a server, a computing system, a communication device, a smartphone, a tablet computer, a smart television, a processor (e.g. digital signal processor (DSP), a microcontroller, a field programmable gate array (FPGA), an application specific integrated circuit (ASIC), etc.), a group of multiple physical machines sharing performance of various tasks, virtual servers co-residing on a single physical machine, any other electronic computing device, and/or any combination thereof.

The operations in accordance with the teachings herein may be performed by a computer specially constructed for the desired purposes or by a general-purpose computer specially configured for the desired purpose by a computer program stored in a non-transitory computer readable storage medium. The term “non-transitory” is used herein to exclude transitory, propagating signals, but to otherwise include any volatile or non-volatile computer memory technology suitable to the application.

As used herein, the phrase “for example,” “such as”, “for instance” and variants thereof describe non-limiting embodiments of the presently disclosed subject matter. Reference in the specification to “one case”, “some cases”, “other cases” or variants thereof means that a particular feature, structure or characteristic described in connection with the embodiment(s) is included in at least one embodiment of the presently disclosed subject matter. Thus, the appearance of the phrase “one case”, “some cases”, “other cases” or variants thereof does not necessarily refer to the same embodiment(s).

It is appreciated that, unless specifically stated otherwise, certain features of the presently disclosed subject matter, which are, for clarity, described in the context of separate embodiments, may also be provided in combination in a single embodiment. Conversely, various features of the presently disclosed subject matter, which are, for brevity, described in the context of a single embodiment, may also be provided separately or in any suitable sub-combination.

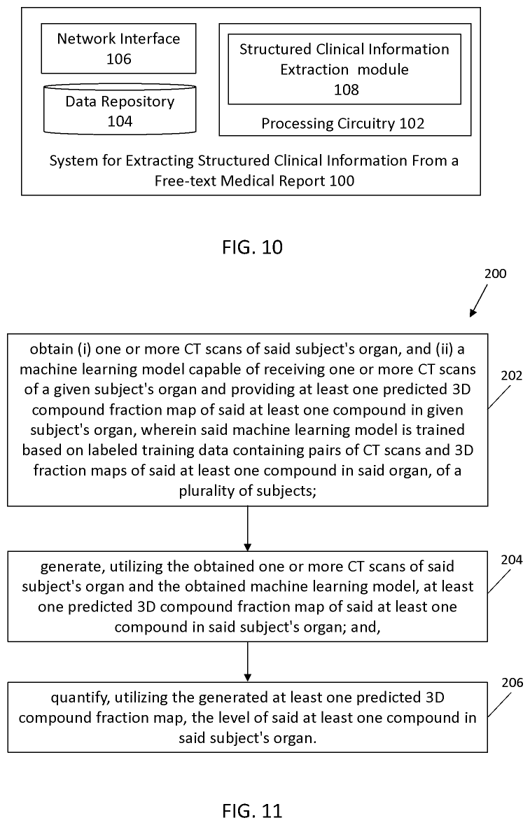

In embodiments of the presently disclosed subject matter, fewer, more and/or different stages than those shown in FIG. 11 may be executed. In embodiments of the presently disclosed subject matter one or more stages illustrated in FIG. 11 may be executed in a different order and/or one or more groups of stages may be executed simultaneously. Each module in FIG. 10 can be made up of any combination of software, hardware and/or firmware that performs the functions as defined and explained herein. The modules in FIG. 10 may be centralized in one location or dispersed over more than one location. In other embodiments of the presently disclosed subject matter, the system may comprise fewer, more, and/or different modules than those shown in FIG. 10.

Any reference in the specification to a method should be applied mutatis mutandis to a system capable of executing the method and should be applied mutatis mutandis to a non-transitory computer readable medium that stores instructions that once executed by a computer result in the execution of the method.

Any reference in the specification to a system should be applied mutatis mutandis to a method that may be executed by the system and should be applied mutatis mutandis to a non-transitory computer readable medium that stores instructions that may be executed by the system.

Any reference in the specification to a non-transitory computer readable medium should be applied mutatis mutandis to a system capable of executing the instructions stored in the non-transitory computer readable medium and should be applied mutatis mutandis to method that may be executed by a computer that reads the instructions stored in the non-transitory computer readable medium.

By way of introduction, the presently disclosed subject matter relates to a novel prompt-based natural language processing (NLP) framework tailored for the medical domain. This framework, hereinafter referred to as SMP-BERT, leverages advanced prompt learning techniques that exploit the structured nature of medical reports to improve both model learning and performance. By doing so, it addresses common challenges such as data scarcity and class imbalance, while also enhancing privacy safeguards by minimizing the need for direct exposure to sensitive data during training.

### SMP-BERT Framework

### Section Matching Prediction

In alignment with the above, the presently disclosed subject matter introduces a Section Matching Prediction (SMP) task, designed specifically for analysis of medical reports, such as, but not limited to, radiology reports. These reports typically contain structured sections, notably “Findings” and “Impression”. The “Findings” section provides detailed observations from radiological examinations, whereas the “Impression” section offers crucial observations and their summarized interpretations.

Inspired by the Next Sentence Prediction (NSP) approach, the SMP task considers “Findings” as the first segment and “Impression” as the follow-up. During training, “Impression” sections are accurately matched with their corresponding “Findings” counterparts in 50% of the instances (Match), and randomly mismatched in the remaining 50% (NotMatch), forming a binary classification objective.

It should be noted that the above serves as a mere example provided solely for illustrative purposes and not intended to limit the scope of the presently disclosed subject matter. Alternative match ratios or training methodologies may also be applicable, mutatis mutandis.

Let M denote a model trained on radiology reports. The model is trained on the SMP task, where x^{F} and x^{I} represent the “Findings” and “Impression” sections, respectively. The model's input takes the following form:

$${x}_{input} = [ CLS ] {x}_{i}^{F} [ SEP ] {x}_{i}^{I} [ EOS ]$$

Let

$${q}_{ℳ} ( {𝓃}_{k} \vert {x}_{i}^{F} , {x}_{i}^{I} )$$

denote the output probability from the model's SMP head based on the input, where n E {Match, NotMatch}. The scores s are computed by:

S=Wₛₘₚ(Tanh(Wh_{[CLS]}+b))
where h_{[CLS]} represents the hidden vector of the special token [CLS] and Wₛₘₚ is the SMP head matrix. The output probability is calculated using the softmax function:

$${q}_{ℳ} ( {𝓃}_{k} \vert {x}_{i}^{F} , {x}_{i}^{I} ) = \frac{exp s ( {𝓃}_{k} \vert {x}_{i}^{F} , {x}_{i}^{I} )}{{\sum }_{n} exp s ( 𝓃 \vert {x}_{i}^{F} , {x}_{i}^{I} )}$$

This training procedure, optimized using a cross-entropy loss function, enables the model to effectively learn and evaluate the logical consistency between Findings and Impression sections. During inference, this learned capability can be harnessed to generate prompts that directly target the presence or absence of specific findings within the reports.

Inference with SMP-BERT

During the inference stage, SMP-BERT leverages its pre-trained understanding of the semantic relationship between the between “Findings” and “Impression” sections. In this phase, the “Impression” section is substituted with a prompt corresponding to the presence or absence of a clinical finding.

By analyzing the combination of the “Findings” section and the prompt, SMP-BERT assigns a higher probability to “Match”″ when the prompt is semantically aligned with the content of the “Findings” section.

The input for inference is structured as follows:

$${x}_{input} = [ CLS ] {x}_{i}^{F} [ SEP ] {x}_{i}^{I} [ EOS ]$$

Here, p^{j} represents the prompt corresponding to the j′th label (presence/absence of a finding).

The template T combines the report's findings section (xᵢ^{F}) with generalized prompt: T(x)=[CLS]x^{F} [SEP] There {is/isn't} {finding} in the {organ} [EOS]. This approach maps labels to prompts of varying lengths. A verbalizer function
f: Y→P associates each label yⁱ∈Y with its corresponding prompt pj∈P. For example, let pⁱ=“There is narrowed lumen in the Ileum” and p^{k}=“There is not narrowed lumen in the Ileum” then, the prediction for report xᵢ regarding narrowed lumen in the Ileum would be:

$$arg max ( {q}_{ℳ} ( Match \vert {x}_{i}^{F} , {p}^{k} ) , {q}_{ℳ} ( Match \vert {x}_{i}^{F} , {p}^{j} ) ) .$$

### SMP-Tuning

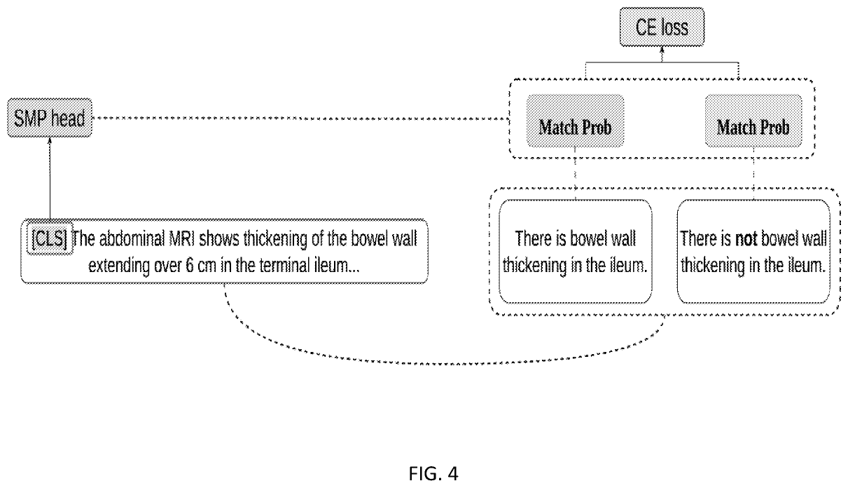

The SMP-tuning process is visualized in FIG. 4 and conducted similarly to the approach of NSP-tuning from NSP-BERT.

Generally, this process extends the SMP pre-training, but instead of using actual “Impression” sections, it relies on prompts derived from annotated reports. Given a sample i with its reference label yᵢ⁺, a positive instance was defined as (T(xᵢ, yᵢ⁺), Match) and for each label yᵢ⁻ that does not match the reference label, negative instances were defined as {(T(xᵢ,yᵢ), NotMatch)}_{yi}_{^{−}}_{∈Y\{yi}_{⁺}}, where Y is the set of all possible labels.

This constructed data sums up to (n_samples*n_phenotypes*n_labels) instances and then used to fine-tune the model, leveraging the initialized weights from the SMP pre-training phase.

### Experiments

The following experiments are provided solely for illustrative purposes and should not be construed as limiting the scope of the disclosed subject matter in any way. These experiments are intended to offer individuals skilled in the art a comprehensive understanding of how to make and use the described invention, ensuring full and clear disclosure. They are not, however, exhaustive or restrictive. The scope of the invention is not confined to these experiments, nor should these specific experiments imply that they represent the entirety of testing or applications. Rather, the experiments serve to highlight various aspects of the invention, and other modifications, variations, and adaptations, as 10 would be apparent to those skilled in the art, remain fully within the intended scope of the invention.

### Data

The study's dataset consists of radiology reports from three medical institutions, collected over a period extending from 2010 to 2023. It includes 9,683 free-text reports (one for each visit) and covers 8093 distinct patients. Since this dataset is confidential, it has not been used in prior model performance evaluations. Ethics approval for the use of this dataset was granted by the Shaare Zedek Medical Center Institutional Review Board (Helsinki) committee.

For the purpose of this study, a subset of 700 reports were manually annotated to idnetify the presence or absence of certain phenotypes in various organs according to the Consensus Recommendations of the American Gastroenterological Association and the Society for Abdominal Radiology. The targeted organs included the jejunum, ileum, cecum, colon, sigmoid, and rectum. The annotated findings encompassed: bowel wall thickening, hyper-enhancement, pre-stenotic dilatation, narrowed lumen, restricted diffusion, and comb sign.

Since the radiology reports were written in free-text format, they were segmented into “Findings” and “Impression” sections using keywords like “In summary:”.

### Experimental Setup

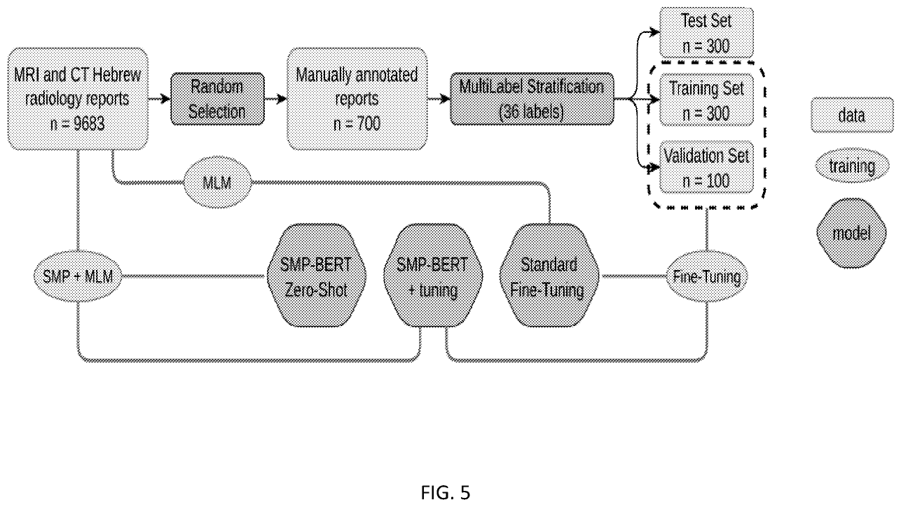

To facilitate a robust evaluation, the dataset was split into three distinct sets using a multi-label stratification. The division was as follows (see FIG. 5): (i) training (300 reports), (ii) validation (100 reports), and (iii) test (300 reports).

This stratification was crucial to maintain representative distributions of labels across the sets, considering the significant class imbalance present in the majority of labels.

The goal was to compare the performance of our method against standard fine-tuning and assess the advantages of adding the SMP-tuning step on top of the zero-shot approach.

The foundation of the models is the Hebrew ROBERTa (HeRo) model, initially pre-trained on the HeDC4 corpus, a comprehensive Hebrew language corpus. The model was further pre-trained on all our radiology reports using the Masked Language Modeling (MLM) task, since there are no other open medical large corpora for Hebrew.

Experiments were conducted using three models:
Standard Fine-tuning: This model was finetuned directly for multi-label classification for all phenotypes.
SMP-BERT Zero-Shot: This model was further pre-trained on all radiology reports using the SMP task. Inference was executed using the SMP-BERT methodology mentioned in the Inference section.
SMP-BERT+tuning: Like the zero-shot model, this model underwent pre-training with the SMP task on all radiology reports. Additionally, it was trained further using SMP-tuning to optimize its performance.

In addition, the impact of training set size was evaluated by training the models on datasets of varying sizes (50 to 300 reports), in order to examine how the quantity of training data influences model performance and generalization to unseen data.

Furthermore, an ablation study was further conducted to isolate and assess the individual contributions of MLM and SMP pre-training tasks to the overall effectiveness of the model

The initial goal was to compare our method with open-source generative LLMs like Llama 2. However, currently available open-source LLMs are not optimized for low-resource languages such as Hebrew, which made the comparison infeasible.

Given the inherent class imbalance in the dataset, where most labels have a low number of positive samples, the models were primarily evaluated using the F1-score alongside the AUC metric. The F1-score considers both precision and recall, making it well-suited for imbalanced datasets. Additionally, the Interquartile Range (IQR), along with the scores, were reported to provide insight into the variability and distribution of model performance across different labels.

All experiments were conducted using a single NVIDIA RTX A6000 GPU, with each experiment taking approximately 1-3 hours.

### Hyper-Parameters

For the SMP-BERT+tuning configuration, the model was trained for 6 epochs on the constructed dataset, comprising 21,600 instances (calculated as 300 reports×36 labels×2 classes). In contrast, the Standard Fine-Tuning model was trained for 120 epochs on the original dataset of 300 reports.

For both training regimes, the learning rate was set to 2e-5 with linear decay, and the batch size was fixed at 24.

### Results

To account for the inherent class imbalance in the dataset, the analysis was focused on phenotypes with at least 10 positive samples, ensuring the reliability of the findings.

The evaluation across three distinct model configurations highlighted the superior performance of the SMP-BERT+tuning approach in extracting phenotypic information from CD radiology reports. The SMP-BERT+tuning model achieved the highest median AUC of 0.99 (IQR 0.98-0.99), outperforming the Standard Fine-tuning model's median AUC of 0.94 (IQR 0.92-0.96) and the SMP-BERT Zero-Shot model's median AUC of 0.88 (IQR 0.81-0.91). For F1-score evaluations, the SMP-BERT+tuning model again leads with a median score of 0.84 (IQR 0.76-0.94), which is substantially higher than the scores of the Standard Fine-tuning model (0.34, IQR 0.22-0.85) and the SMP-BERT Zero-Shot model (0.58, IQR 0.55-0.62). A comprehensive breakdown of these results, including F1 and AUC scores for individual phenotypes, is detailed in the FIG. 8.

Further analysis presented in FIG. 7 of model performance relative to the count of positive instances exhibited the strength of SMP-BERT+tuning, particularly for labels with sparse positives in the training set. For example, with only 19 positive cases for “Rectum Bowel Wall Thickening,” SMPBERT+tuning achieved a significantly higher F1-score (0.74) compared to the standard model (0.1). This demonstrates its superior ability to generalize well from limited data.

However, both models performed well when dealing with abundant positive instances. For example, with 137 positives for “Ileum Bowel Wall Thickening” (almost half the dataset), both models achieved good results, with SMP-BERT+tuning maintaining a decent gap (F1-score 0.97 vs. 0.915 for the standard model).

The graph shown in FIG. 7 indicates that the performance gap between the models decreases with an increase in the number of positive instances. This suggests that while SMP-BERT+tuning shines with limited data, it still performs better when more data is available.

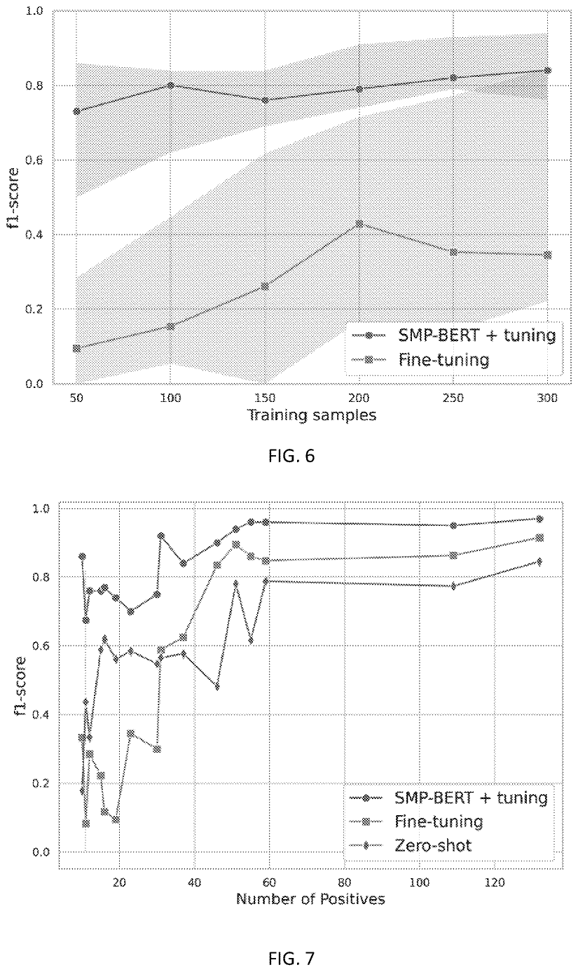

Also analyzed was the effect of the size of the training set on model performance. As shown in FIG. 6, the SMP-BERT+tuning model exhibits superior adaptability. Notably, it achieves good performance even with limited training data (50-100 samples). The Standard Fine-tuning model exhibits a trend of broadening IQRs and decrease of median score.

This could suggest an improving performance for common phenotypes (like Ileum Bowel Wall Thickening) but potentially decreasing performance for rarer ones due to increased data imbalance.

### Ablation Study

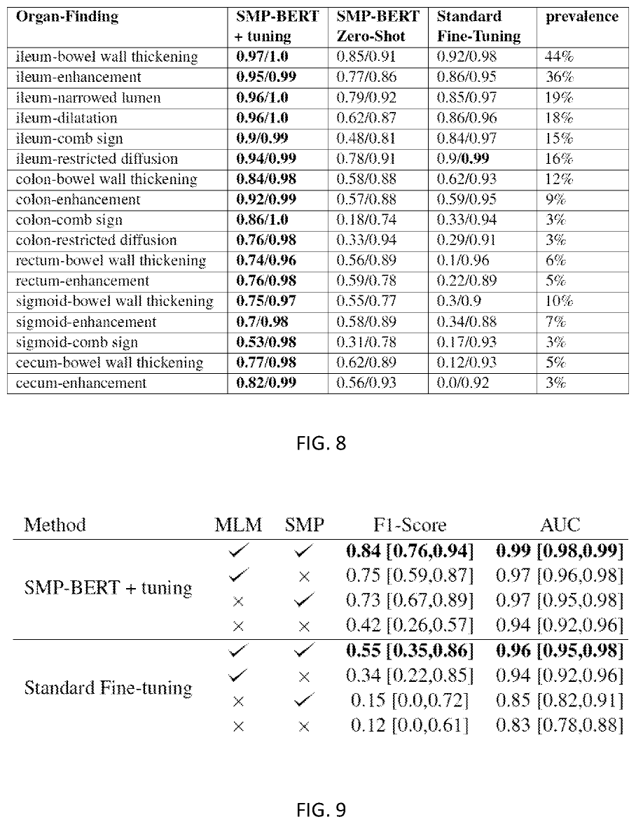

As evidenced in FIG. 9, both pre-training tasks, MLM and SMP, significantly contribute to optimizing the performance of SMP-BERT. Moreover, it appears that standard fine-tuning benefits from the inclusion of the SMP task.

Attention is now drawn to FIG. 10, depicting a block diagram schematically illustrating one example of the system for extracting structured clinical information from a free-text medical report 100, in accordance with the presently disclosed subject matter.

In accordance with the presently disclosed subject matter, the system for extracting structured clinical information from a free-text medical report 100 (also interchangeably referred to herein as “system 100”) may comprise a network interface 106. The network interface 106 (e.g., a network card, a Wi-Fi client, 3G/4G client, or any other component), enables system 100 to communicate over a network with external systems and handles inbound and outbound communications from such systems. For example, system 100 may receive, through network interface 106, one or more text segments from one or more medical reports written in at least one natural language.

System 100 may further comprise or be otherwise associated with a data repository 104 (e.g., a database, a storage system, a memory including Read Only Memory-ROM, Random Access Memory-RAM, or any other type of memory, etc.) configured to store data. Some examples of data that may be stored in the data repository 104 include:
One or more text segments from one or more medical reports written in at least one natural language;
One or more machine learning models capable of (i) receiving a given text segment from a given medical report written in a given natural language, and (ii) providing given structured clinical information indicating presence or absence of given clinical phenotypes;
One or more clinical phenotypes associated with one or more medical reports of one or more patients;
Structured clinical information indicating presence or absence of clinical phenotypes;
Labeled training data containing historical medical reports; etc.

Data repository 104 may be further configured to enable retrieval and/or update and/or deletion of the stored data. It is to be noted that in some cases, data repository 104 may be distributed, while the system 100 has access to the information stored thereon, e.g., via a wired or wireless network to which system 100 is able to connect (utilizing its network interface 106).

System 100 further comprises processing circuitry 102. Processing circuitry 102 may be one or more processing units (e.g., central processing units), microprocessors, microcontrollers (e.g., microcontroller units (MCUs)) or any other computing devices or modules, including multiple and/or parallel and/or distributed processing units, which are adapted to independently or cooperatively process data for controlling relevant system 100 resources and for enabling operations related to system's 100 resources.

The processing circuitry 102 comprises a structured clinical information extraction module 108, configured to perform an extraction process, as further detailed herein, inter alia with reference to FIG. 11.

Turning to FIG. 11 there is shown a flowchart illustrating one example of operations carried out by the system for extracting structured clinical information from a free-text medical report 100, in accordance with the presently disclosed subject matter.

Accordingly, the system for extracting structured clinical information from a free-text medical report 100 (also interchangeably referred to hereafter as “system 100”) may be configured to perform an extraction process 200, e.g., using structured clinical information extraction module 108.

For this purpose, system 100 obtains: (a) a text segment from said medical report written in at least one natural language, and (b) a machine learning model capable of (i) receiving a given text segment from a given medical report written in at least one given natural language, and (ii) providing given structured clinical information indicating presence or absence of given clinical phenotypes (block 202).

Once obtained, system 100 extracts, based on the text segment and utilizing the machine learning model, structured clinical information indicating presence or absence of clinical phenotypes (block 204).

In some cases, the machine learning model may be pre-trained, for example, using a Section Matching Prediction (SMP) task, where the model is trained using labeled training data including pairs of text segments from historical medical reports, labeled as either “Match”, indicating that said pair is from the same medical report, or “Not Match”, indicating that said pair is not from the same medical report.

It is to be of note that the above presentation of “Match” and “Not Match” serves as a mere example not intended in any way to limit the scope of the claimed subject matter, and that other forms of presentations may also be applicable, mutatis mutandis

In some cases, following the SMP task, the machine learning model may be further trained to identify semantic agreement between text segments and prompts by, for example, evaluating which of two opposing queries, one indicating presence and one indicating absence of a specific clinical phenotype, yields a higher match probability.

It is to be of note that the above manner serve as mere examples, not intended in any way to limit the scope of the presently disclosed subject matter, and that other manners, alternatively or additionally to the above, may also be applicable.

In some cases, further to the obtaining of the text segment, system 100 may obtain, either in a hierarchical or flat fashion, one or more prompts corresponding to one or more clinical queries related to one or more clinical phenotypes. In such cases, the machine learning model may be configured to output the given structured clinical information based on the semantic agreement between the text segment and the one or more prompts, such that the structured clinical information is further based on said prompts.

In some cases, prior to the SMP task, the machine learning model may be pre-trained, for example, using a Masked Language Modeling (MLM) task, where one or more tokens in one or more training medical reports are replaced with one or more mask tokens, and the model is trained to predict the masked tokens based on surrounding contextual tokens.

It is to be of note that the above manner serve as mere examples, not intended in any way to limit the scope of the presently disclosed subject matter, and that other manners, alternatively or additionally to the above, may also be applicable.

In some cases, the natural language may be a low-resource language, such as Hebrew, Amharic, Yiddish, and the like.

In some cases, the at least one natural language may include two or more languages. For example, the medical report be primarily written in Hebrew, while also incorporating English terms for medical concepts or terminology.

It is to be noted, with reference to FIG. 11, that some of the blocks can be integrated into a consolidated block or can be broken down to a few blocks and/or other blocks may be added. It is to be further noted that some of the blocks are optional. It should be also noted that whilst the flow diagram is described also with reference to the system elements that realizes them, this is by no means binding, and the blocks can be performed by elements other than those described herein.

It is to be understood that the presently disclosed subject matter is not limited in its application to the details set forth in the description contained herein or illustrated in the drawings. The presently disclosed subject matter is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the present presently disclosed subject matter.

It will also be understood that the system according to the presently disclosed subject matter can be implemented, at least partly, as a suitably programmed computer. Likewise, the presently disclosed subject matter contemplates a computer program being readable by a computer for executing the disclosed method. The presently disclosed subject matter further contemplates a machine-readable memory tangibly embodying a program of instructions executable by the machine for executing the disclosed method.

1. A method for extracting structured clinical information from a free-text medical report comprising:
obtaining: (a) a text segment from said medical report written in at least one natural language, and (b) a machine learning model capable of (i) receiving a given text segment from a given medical report written in at least one given natural language, and (ii) providing given structured clinical information indicating presence or absence of given clinical phenotypes; and,
extracting, based on said text segment and utilizing said machine learning model, structured clinical information indicating presence or absence of clinical phenotypes;
wherein said machine learning model is pre-trained using labeled training data including pairs of text segments from historical medical reports, labeled as either “Match”, indicating that said pair is from the same medical report, or “Not Match”, indicating that said pair is not from the same medical report, and wherein, following said pre-training, said machine learning model is further trained to identify semantic agreement between text segments and prompts by evaluating which of two opposing queries, one indicating presence and one indicating absence of a specific clinical phenotype, yields a higher match probability.

2. The method of claim 1, wherein said machine learning model is pre-trained using a Section Matching Prediction (SMP) task.

3. The method of claim 1 further comprising obtaining one or more prompts corresponding to one or more clinical queries related to one or more clinical phenotypes, wherein the machine learning model is configured to receive said one or more prompts, along with said text segment, and to provide said given structured clinical information based on the semantic agreement between the text segment and the one or more prompts, such that said structured clinical information is further based on said prompts.

4. The system of claim 3, wherein said one or more prompts are provided in a hierarchical fashion.

5. The method of claim 2, wherein, prior to said SMP task, said machine learning model is pre-trained using a Masked Language Modeling (MLM) task, where one or more tokens in one or more training medical reports are replaced with one or more mask tokens, and the model is trained to predict the masked tokens based on surrounding contextual tokens.

6. The method of claim 1, wherein said medical report is a radiology report.

7. The method of claim 6, wherein each pair of said pairs of text segments involves a first text segment, corresponding to a “Findings” section, and a second text segment, corresponding to an “Impression” section.

8. The method of claim 7, wherein said semantic is determined between a “Findings” section and a corresponding prompt.

9. The method of claim 1, wherein said at least one natural language is a low-resource language.

10. The method of claim 1, wherein said at least one natural language includes two or more languages.

11. A system for extracting structured clinical information from a free-text medical report, the system comprising a processing circuitry configured to:
obtain: (a) a text segment from said medical report written in a natural language, and (b) a machine learning model capable of (i) receiving a given text segment from a given medical report written in a given natural language, and (ii) providing given structured clinical information indicating presence or absence of given clinical phenotypes; and,
extract, based on said text segment and said machine learning model, structured clinical information indicating presence or absence of clinical phenotypes;
wherein said machine learning model is pre-trained using labeled training data including pairs of text segments from historical medical reports, labeled as either “Match”, indicating that said pair is from the same medical report, or “Not Match”, indicating that said pair is not from the same medical report, and wherein, following said pre-training, said machine learning model is further trained to identify semantic agreement between text segments and prompts by evaluating which of two opposing queries, one indicating presence and one indicating absence of a specific clinical phenotype, yields a higher match probability.

12. The system of claim 11, wherein said machine learning model is pre-trained using a Section Matching Prediction (SMP) task.

13. The system of claim 11 further comprising obtaining one or more prompts corresponding to one or more clinical queries related to one or more clinical phenotypes, wherein the machine learning model is configured to receive said one or more prompts, along with said text segment, and to output said given structured clinical information based on the semantic agreement between the text segment and the one or more prompts, such that said structured clinical information is further based on said prompts.

14. The system of claim 12, wherein, prior to said SMP task, said machine learning model is pre-trained using a Masked Language Modeling (MLM) task, where one or more tokens in one or more training medical reports are replaced with one or more mask tokens, and the model is trained to predict the masked tokens based on surrounding contextual tokens.

15. The system of claim 11, wherein said medical report is a radiology report.

16. The system of claim 15, wherein each pair of said pairs of text segments involves a first text segment, corresponding to a “Findings” section, and a second text segment, corresponding to an “Impression” section.

17. The system of claim 17, wherein said semantic is determined between a “Findings” section and a corresponding prompt.

18. The system of claim 11, wherein said natural language is a low-resource language.

19. The system of claim 11, wherein said at least one natural language includes two or more languages.

20. A non-transitory computer readable storage medium having computer readable
program code embodied therewith, the computer readable program code, executable by at least one processor to perform a method for extracting structured clinical information from free-text medical reports, the method comprising:
obtaining: (a) a text segment from said medical report written in a natural language, and (b) a machine learning model capable of (i) receiving a given text segment from a given medical report written in a given natural language, and (ii) providing given structured clinical information indicating presence or absence of given clinical phenotypes; and,
extracting, based on said text segment and said machine learning model, structured clinical information indicating presence or absence of clinical phenotypes;
wherein said machine learning model is pre-trained using labeled training data including pairs of text segments from historical medical reports, labeled as either “Match”, indicating that said pair is from the same medical report, or “Not Match”, indicating that said pair is not from the same medical report, and wherein, following said pre-training, said machine learning model is further trained to identify semantic agreement between text segments and prompts by evaluating which of two opposing queries, one indicating presence and one indicating absence of a specific clinical phenotype, yields a higher match probability.
